(19) United States
(12) Patent Application Publication
(10) Pub. No.: US 2010/0003174 A1
(43) Pub. Date: Jan. 7, 2010

(54) Golden touch container system

(76) Inventor: Ahmed Salih Mohamed, Jersey City, NJ (US)

Correspondence Address: AHMED SALIH MOHAMED;SUITE 1E, 7-11 TONNELE AVE., JERSEY CITY, NJ, 07306, US

(21) Appl. No.: 12/456,305

(22) Filed: Jun. 15, 2009

Related U.S. Application Data
(60) Provisional application No. 61/129,561, filed on Jul. 7, 2008.

(51) Int. Cl. A61L 2/00

(52) U.S. Cl. 422/295

(57) ABSTRACT

The new thing in this invention is using a mechanical power or electrical power interacting with pressurized liquid or liquid gas using touchable surfaces, door knobs handles as a vain to get the sanitizer out in form of gas spray etc. directly and automatically to the human hands.

### CROSS REFERENCE

U.S.A provisional patent date of filling Jul. 7, 2008

Cross-Reference U.S.A. provisonal patent

The golden touch container system is versatile device built to replace the existing door knobs and handles for purpose of sanitization, hygiene and perfume. The usage for the golden touch container system (G.T.C.S) for homes, hospitals, schools and public areas. The (G.T.C.S) consist of containers, aerosols, bottles and tanks made of plastic, metal, fiber carbon, wood and glass. These said (G.T.C.S) is to be filled and refilled with chemical sanitizer or organic sanitizer or any hygienic product that's suitable. This will be under gas pressure inside the container to ease puffing, streaming and spraying from the (G.T.C.S) through the door knobs and handles, when touching or turning the said door knobs or handles whether is electronically, electrically, or manually for the purpose of automatic hand sanitizing. The reason for that is to eliminate germs, virus on hands or door knob.

### BACKGROUND OF THE INVENTION

This invention (G.T.C.S) can be best described as a surgical and pinpointing automatic hand sanitizer device. So it's classified as a hand sanitizer. The prior art was a bar of soap liquid sanitizer in a small container or in hospitals wall-mounted device which can be forgotten or skipped by careless individuals.

### BRIEF SUMMARY OF THE INVENTION

Because the hand sanitizer is built in the door knobs or handles, which means you must turn or turn it to get threw the door which will make it an advantages over the priors arts which you can forget or skip.

### BRIEF DESCRIPTION OF THE SEVERAL VIEWS OF DRAWING

FIG. 1 shows the new modified home door.

FIG. 1 arrow number one shows the new modified door knob

FIG. 1 arrow number two shows the hallow area where in some part of the device can be installed

FIG. 1 arrow #3 shows the hole for movements of mechanical rod

FIG. 1 arrow #4 shows the upper cover part of the door opened by means of hinges.

FIG. 2 arrow #2 shows the female part of the new door knobs

FIG. 2 arrow #5 refill cap

FIG. 2 arrow #6 the new modified door knobs tongues hole

FIG. 3 arrow #7 the mechanical doors movement air pumps

FIG. 3 arrow #8 the mechanical semi-flexible rod which pushes the air pumps

FIG. 3 arrow #9 shows part of air bleeding holes

FIG. 4 arrow #8 shows the flexible rod attached to the main door frame

FIG. 4 arrow #10 shows the flexible rods holder which is attached to the main door frame which only move can slightly move horizontally to smooth the mechanical flexible rod in and out.

FIG. 4 arrow number eleven shows a metallic bar by which the flexible rod attach to

FIG. 5 arrow number twelve shows part of a refill additional hose extension

FIG. 5 arrow #13 shows main tank or container

FIG. 5 arrow#14 shows door moving edge when opening and closing

FIG. 5 arrow#15 shows a micro electrical air compressor attached to the doors base

FIG. 6 arrow #1 male door knobs

FIG. 6 arrow #16 shows the electrical micro air compressor extension hose

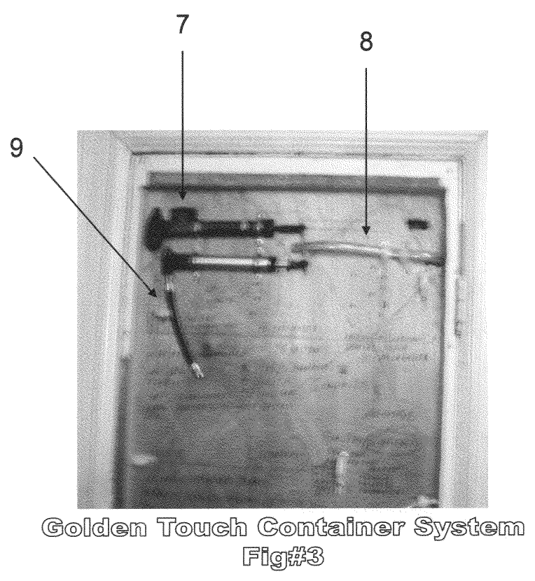

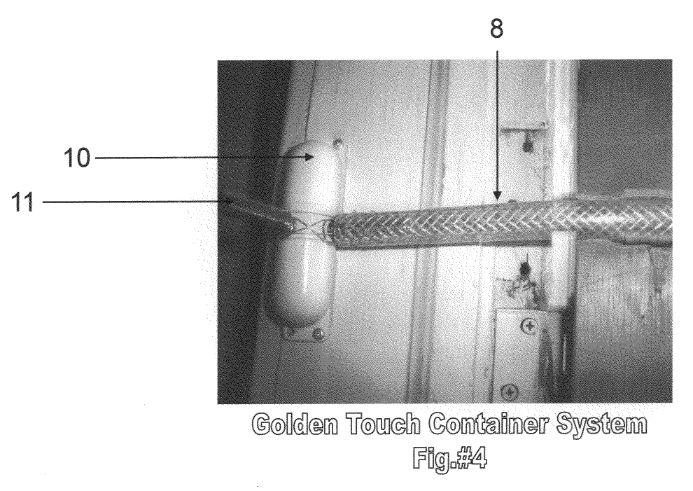

FIG. 2 arrow #17 holes and valves connection

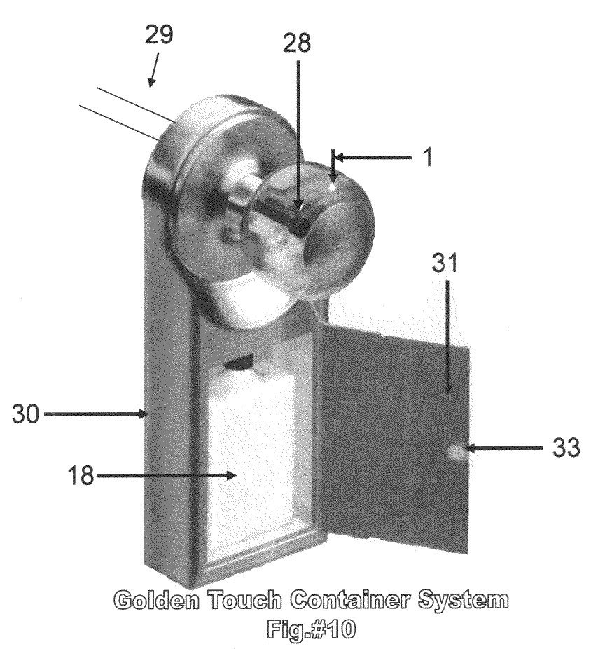

FIG. 6 arrow #18 shows a tank or container

FIG. 6 arrow #19 shows the tank or container support shelf

FIG. 6 arrow #20 door knob valve

FIG. 6 #21 additional refill valves

FIG. 7 arrow #1 shows male door knobs or handles

FIG. 7 arrow #22 shows the head of the hammer which pushes the nozzles button to release the spray on/off switches in case of electrical (G.T.C.S) device

FIG. 7 arrow #23 nozzle push button or negative electrical circuits

FIG. 7 arrow #24 rear door knob nozzle

FIG. 8 arrow #1 shows L shaped door knob

FIG. 8 arrow #26,27 show the hollow door handle with the small holes for spraying the hand sanitizer

FIG. 9 arrow #28 shows upwards nozzle that is spraying out the hand sanitizer

FIG. 9 arrow #29 rear means of nozzle

FIG. 10 arrow #30 doors knobs frame fixture

Arrow 31 doors container knob lid or cover

Arrow 32 means of key locking system

Arrow 1 front door knob

Arrow 18 container with sanitizer or perfumes

Arrow 28 upwards nozzle sanitizer

Arrow 29 means of rear nozzle

### DETAILED DESCRIPTION OF THE INVENTION

The Golden Touch container system is a very unique, economic, and healthy it is a very practical invention, by looking deep into the existing technologies as a prior art it is very poor, and out dated also incomparable to the (G.T.C.S). for example door knobs are well known as germ and virus collectors, especially that one for public bathrooms and your fears to touch it, lets assume that you clean your hand inside the bathroom with a bar a soap or liquid soap mounted on the walls or the sanitizer device like those mounted to the hospital walls from inside, still you are not show the person before you left germs or virus on the door knob now here comes (G.T.C.S) to give you assurance, tranquility and a peace of mind. The technologies and the arts of the (G.T.C.S) is made possible basically human hygiene's putting in consideration the quality and quantity of the sanitizers, perfumes from chemical sanitizers or organic sanitizer to avoid allergies to skin and respiratory system human begins and other animals, putting also in consideration environmental issues, safety and concerns, toxic fumes, suffocation, fire hazards and federal and state laws ((domestic and industrial safety guidelines)). What is the(G.T.C.S) consist of? The (G.T.C.S) basically, containers, tanks, air pumps, air compressor, straws, nozzles and electronic motion sensors to sense approaching hand to the door knobs, handles, rechargeable batteries, clamps, hoses and extensions pressure valves to control the pressure inside the tank to avoid explosion. How does (G.T.C.S) operate? The (G.T.C.S) operates in so many different ways for example ?1. manually or mechanically by turning the door knob by pushing or pulling the door
2. Electrically or semi electrically by touching the door knob you are connecting and disconnecting electrical circuits.
3. Electronically by means of inferred beams or other electronic circuit beams operating system (motion sensors).

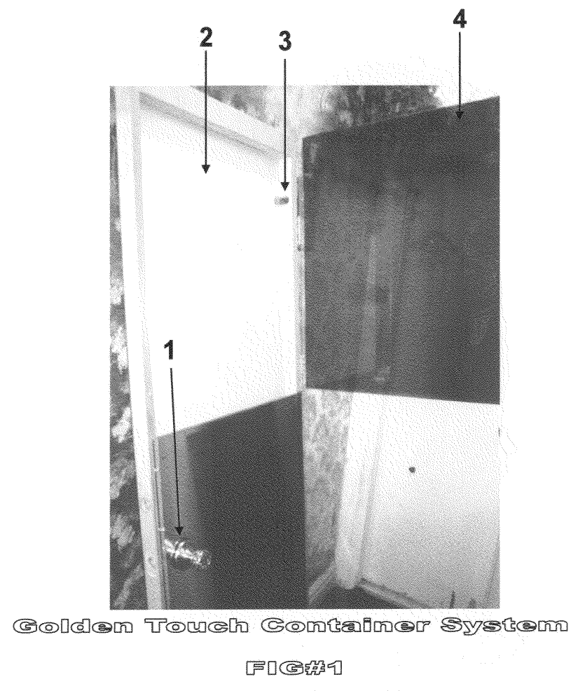

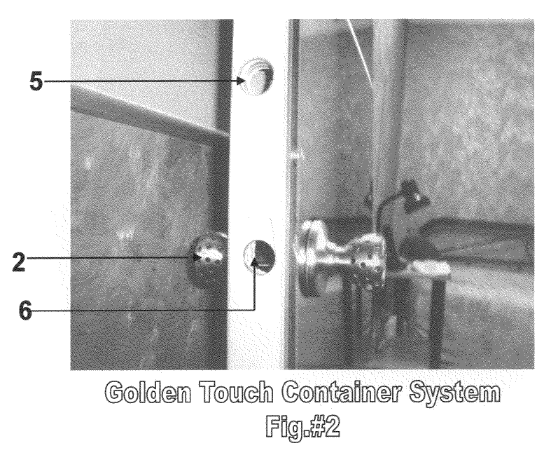

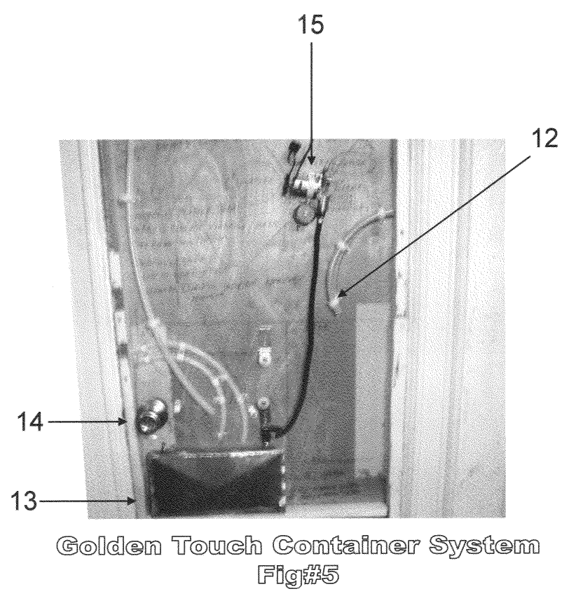

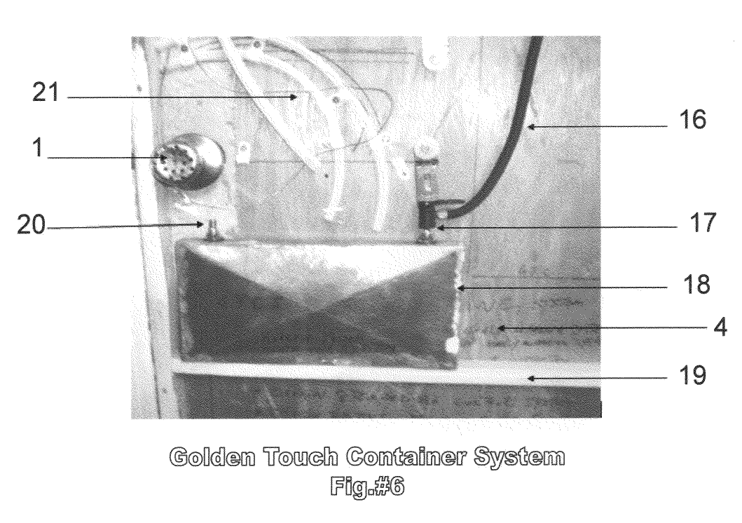

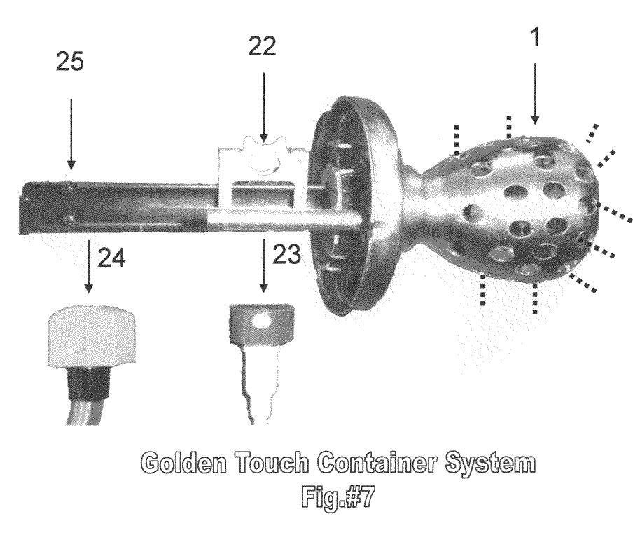

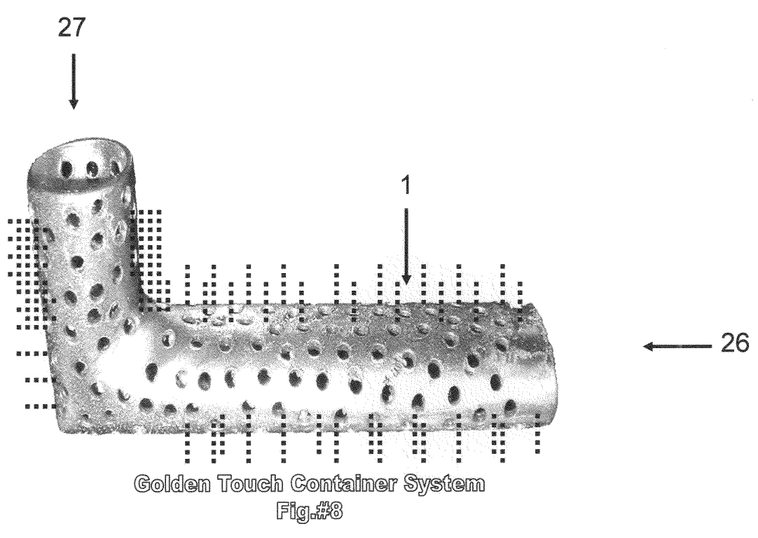

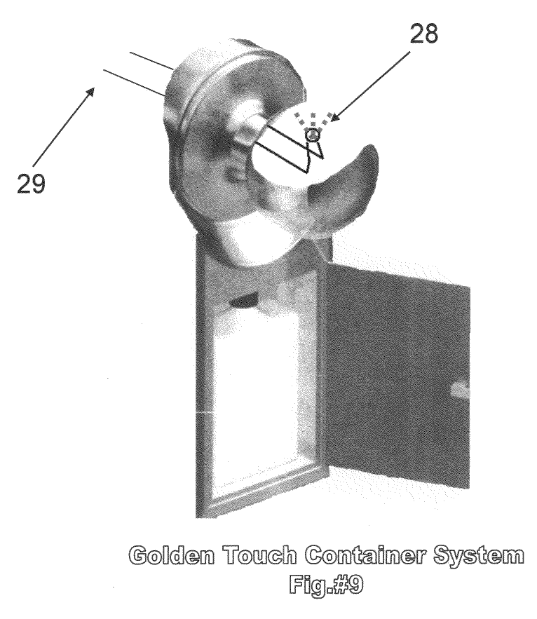

By referring to FIG. 1 is a modified wooden door 24×77×2 inches. Hollow from inside please see FIG. 1 arrow #2 shows the hollow interior of the said door. Arrow #3 a hole through which the said flexible rod in FIG. 4 arrow #8 to allow the mechanical air pumps movement, when operating the said door in FIG. 1, in FIG. 1 arrow #4 shows a carbonated glass doors front cover, upper parts can be open and closed for maintenance, the cover has independent small hinges and screws to be secured. So when you turn the door knob as in FIG. 1 arrow #1 and pull out to enter, actually you are operating the mechanical air pumps in FIG. 3 arrow #7, according to Newton's law for any action there is a reaction. So you are pulling towards yourself, but the rod in FIG. 3 arrow #8 pushes in wards toward the mechanical air pumps arrow #7. Like this you are generating enough air going threw the extension holes #9 in FIG. 3 this air is stored in the aluminum tank which is 12×6×1 inches. As best seen FIG. 6 arrow #18, through the secured holes in FIG. 6 arrow #16, it is very interesting to use modified air pump cylinder position similar to the aluminum tank in FIG. 6 arrow #18 to replace the air pumps in FIG. 3 arrow #7 for much air pumps to be pushed through the system (not used) in this experiment. As you are doing two actions: 1 turning the door knobs, best seen FIG. & arrow #1, this action either way, when you turn the door knobs clockwise or anti clockwise, the head hammer in FIG. 7 arrow #22 will push the nozzle button arrow #23 FIG. 7, which will release the sanitizer (under the pressure) best seen in FIG. 9 arrow #28. By freeing your hand off the door knobs as seen in FIG. 7 this door knob is light duty 400.000 cycles (life span) will go back to its position by helping of fixed spring inside the door knob mechanically. That means the pressure that was applied by your action did release the spray of puffing which will automatically sanitize your hand. Electrically same thing happens as in FIG. 5 arrow #15 shows micro air compressor with pressure reading gages, and electrical switch off/on circuits it is a bell micro compressor, 12 volt dc or ac inverted into dc, micro air cooling fan for the compressor attached. The 12 volt dc micro air compressor is very ideal for vehicles door handles, power steering column and shifting knob. The (G.T.C.S) can be used also in airplanes, ships, trains, boats, escalators, seaports and airports. Where the system differs in electrical power and devices from 1. high speed traffic gates or doors
2. Heavy duty usage doors and gates
3. Medium duty usage doors and gates
4. Light duty usage doors and gates same one used in this experiment. These above devices is different functionality are of a great help to eliminate diseases and sickness especially when there is an outbreak such as H1N1 (swine flu) now a days we thought of it 5 years ago. The (G.T.C.S) can be executed in a fully new door, reengineered, like the one in FIG. 1 or manipulated door kids, like in FIG. 10 where arrow #1 shows the door knob, arrow #28 shows the spring upwards nozzle, arrow #29 rear door knob means arrow #31 the container cover. Arrow #18 shows the container with light pressure (aerosol style). Arrow #30 main door knob frame in FIG. 9 arrow #28 shows clearly the upwards nozzles, downwards, sideways nozzles are not seen. In FIG. 8 arrow #1 shows different shape of door knobs L shaped made of mainly brass and copper, but the interior for all door knobs and handles are coated hard plastic paint to avoid corrosion and decay, while the outside is coated with metallic paint copper brass etc. and it is also showing the spraying condition or mood in FIG. 8 which is done in dots.

1. The golden touch container system is a very unique invention with very distinctive claims in FIG. 7 arrow #1 shows a mesh door knob through which the sanitizer goes in form of gas, spray and stream to make direct contact with your hand.

2. FIG. 7 arrow #22 shows the head hammer which considers being the heart claim of this invention which pushes the nozzles button to allow the spray to go off.

3. FIG. 9 arrow #28 shows the upward spray nozzle by mean which the sanitizer travels from the container to straws not seen the drawings then to the nozzles.

4. FIG. 6 arrow #18 shows a key claim called the container or tank in which the sanitizer pressurized air and straws not seen. Also pressure control valves not seen to control pressure inside containers

5. FIG. 3 arrow #7 shows air pressure pumps and there accessories arrow #8 show flexible rod which pushes the air pumps

6. FIG. 4 arrow #10 the rod fixture attached to the main door frame

7. FIG. 2 arrow #5 shows the refill hole connected to the hoses see FIG. 6 arrow #21
